SEQUENCE LISTING (4 sequences)

SEQ ID NO: 1 - DNA, 27 bp, Artificial
  Synthetic
tgaagagctactgtaatgatcagtcaa

SEQ ID NO: 2 - DNA, 22 bp, Artificial
  Synthetic
agcaagcttgcaaccttaacca

SEQ ID NO: 3 - DNA, 21 bp, Artificial
  Synthetic
cccaggaaagacagcaacctt

SEQ ID NO: 4 - DNA, 21 bp, Artificial
  Synthetic
ttctcacaaccaggccacttg

(19) United States
(12) Patent Application Publication
(10) Pub. No.: US 2006/0292164 A1
(43) Pub. Date: Dec. 28, 2006

(54) Method for preventing rejection of transplanted tissue

(75) Inventor: David A. Horwitz, Los Angeles, CA (US)

Correspondence Address: Richard F. Trecartin;Dorsey & Whitney LLP, Intellectual Property Department, 555 California Street, Suite 1000, San Francisco, CA, 94104-1513, US

(73) Assignee: University of Southern California

(21) Appl. No.: 11/394,761

(22) Filed: Mar. 31, 2006

Related U.S. Application Data
(60) Provisional application No. 60/667,494, filed on Apr. 1, 2005.

(51) Int. Cl. A61K 39/00; A61K 35/14

(52) U.S. Cl. 424/185.1; 424/93.7

(57) ABSTRACT

Methods of preventing rejection of transplanted tissue. Recipient alloactivated regulatory T cells generated ex vivo are introduced into the recipient before transplantation. Donor antigen is introduced into the recipient after transplantation to boost recipient regulatory T cells.

### PRIORITY CLAIM

This application claims priority to, and the benefit of, under 35 U.S.C. § 119(e), U.S. Provisional Application Ser. No. 60/667,494, filed Apr. 1, 2005, which is incorporated in its entirety by reference herein.

### TECHNICAL FIELD

Methods of preventing rejection of transplanted tissue. Recipient alloactivated regulatory T cells generated ex vivo are introduced into the recipient before transplantation. Donor antigen is introduced into the recipient after transplantation to boost recipient regulatory T cells.

### BACKGROUND OF THE INVENTION

Experimental autoimmune and transplant models have shown that several mechanistic approaches that include clonal deletion, anergy, and effector cell regulation can alter T cell alloreactivity and drive the immune system towards one of unresponsiveness (Elster, E. A., et al., Transpl Immunol 13:87 (2004)). There is increasing evidence that CD4+ cells that constitutively express CD25, the alpha chain of the IL-2 receptor, not only have an important role in preventing autoimmunity, but can also prevent graft rejection (Sakaguchi, S., et al., Immunol Rev 182:18 (2001); Piccirillo, C. A. and Shevach, E. M., Semin Immunol 16:81 (2004); Cohen, J. L., et al., J Exp Med 196:401 (2004)). CD4+CD25+ cells with a typical phenotype and suppressive effects occur naturally (Sakaguchi, S., et al., Immunol Rev 182:18 (2001); Piccirillo, C. A. and Shevach, E. M., Semin Immunol 16:81 (2004)), or can be induced peripherally (Yamagiwa, S., et al., J Immunol 166:7282 (2001); Chen, Z. M., et al., Blood 101:5076 (2003)). Endogenous CD4+CD25+ cells can be expanded (Godfrey, W. R., et al., Blood 104:453 (2004)) so that they can be used in clinical trials. Prior studies have shown that peripheral CD4+CD25+ cells that prevent graft rejection can be induced indirectly using non-depleting CD4 and CD8 monoclonal antibodies, co-stimulatory inhibitors, or immunosuppressive drugs (van Maurik, A., et al., J Immunol 169:5401 (2002); Graca, L., Thompson, et al., J Immunol 168:5558 (2002); Taylor, P. A., et al., J Exp Med 193:1311 (2001); Gregori, S., et al., J Immunol 167:1945 (2001)).

The combination of interleukin 2 (IL-2) and transforming growth factor beta (TGF-β) can induce both CD4+ and CD8+ cells to develop potent immunosuppressive activity (Yamagiwa, S., et al., J Immunol 166:7282 (2001); Gray, J. D., et al., J Exp Med 180:1937 (1994); Zheng, S. G., et al., J Immunol 169:4183 (2002); Horwitz, D. A., Semin Immunol 16:135 (2004); Zheng, S. G., et al., J Immunol 172:1531 (2004); Zheng, S. G., et al., J Immunol 172:5213 (2004)). These cytokines induced naive human, alloantigen-stimulated, peripheral blood CD4+ cells to become CD25+ regulatory cells with a surface phenotype and cytokine-independent suppressive effects indistinguishable from natural CD4+CD25+ cells (Yamagiwa, S., et al., J Immunol 166:7282 (2001)). Moreover, these CD4+CD25+ Treg cells are able to induce other CD4+ cells to develop cytokine-dependent suppressive activity in vitro (Zheng, S. G., et al., J Immunol 172:5213 (2004)).

H-2^{d} anti-H-2^{b} Treg cells generated in the presence of IL-2 and TGF-β ex vivo have been used without other immunosuppression to prevent rejection of H-₂^{b} heart transplants. In previous experiments, CD4+ and CD8+ Treg cells were generated by stimulating DBA/2 (H-2^{d}) mouse T cells with C57BL/6 (H-2^{b}) alloantigens in the presence of IL-2 and TGF-β. These Tregs were antigen-specific and prevented a chronic graft-versus-host disease with features of systemic lupus erythematosus in (DBA/2×C57BL/6) F1 mice. Moreover, a single injection of these cells in mice with established disease doubled their survival (Zheng, S. G., et al., J Immunol 172:1531 (2004)).

### SUMMARY OF THE INVENTION

Peripheral blood mononuclear cells (PBMC) from a recipient are stimulated with one or more donor antigens such as donor PBMCs or other donor cells such as spleen cells in the presence of certain messenger proteins. This results in the formation of recipient regulatory T cells that are alloactivated by the donor antigen. These cells are also referred to as donor alloactivated recipient regulatory T cells or recipient Treg cells. Prior to treatment, the PBMCs may be further purified to produce populations of CD4⁺ T cells, CD8⁺ T cells and/or NK-T cells.

The recipient Treg cells are introduced into the recipient prior to transplantation. Pre-transplantation treatment with recipient Treg cells results in the suppression of graft rejection due to an increase in the population of CD25⁺ regulatory T cells. After transplantation, at least one donor antigen is administered to the recipient to boost the population of recipient Tregs. Although the transplant is a source of foreign histocompatability antigens, the appropriate stimulatory antigens may be shed too slowly for the continues growth and function of the Treg.

In still another aspect, recipient Treg cells are introduced into the recipient prior to transplantation. After transplantation, recipient Tregs and at least one donor antigen are administered to the recipient. Alternatively, the recipient Treg cells and donor antigen are introduced into the recipient as pretreatment before transplantation and additional donor antigen is introduced into the recipient after transplantation alone or in combination with recipient Tregs.

Recipient Tregs in combination with donor antigen can be used to prevent rejection of an organ transplant. For example, in the case of a heart transplant, regulatory T cells are prepared using donor antigen and introduced alone or in combination with donor antigen into the recipient. Thereafter, a heart from the donor is transplanted into the recipient. Donor antigen alone or in combination with recipient Tregs are administered to the recipient after heart transplantation. The preferred recipient is a human.

### BRIEF DESCRIPTION OF THE DRAWINGS

FIG. 1 demonstrates that donor anti-H-2^{b}-specific Tregs induced ex-vivo with TGF-β result in long term survival of mismatched allogeneic heart transplants. Regulatory T cells (Tregs) were generated by stimulating DBA/2 (D2, H-2^{d}) T cells with irradiated C57BL/6 (B6, H-2^{b}) non-T cells and IL-2 in the presence of TGF-β (2 ng/ml) for 5-6 days. T cells stimulated with IL-2 only served controls (Tcon). D2 mice that received B6 heart transplants were injected with 10×10⁶ Treg, Tcon, or Treg depleted CD25+ cells IV on days −1 and +5. Six mice were in each group.

FIG. 2 demonstrates that transferred anti-H-2^{b} Tregs induce alloantigen-specific T cell non-responsiveness. Groups of 4 naive DBA/2 mice were injected IV with or without 10×10⁶ D2 Tcon, Treg cells generated as described in FIG. 1. Another non-injected group served as additional controls. One month later the animals were sacrificed and splenic T cells were alloactivated with B6 or third party, C3H (H-2^{k}) stimulator cells in vitro for 4 days. A. Proliferative activity (mean counts per minute±SEM). P values indicate significant differences between mice that received Treg cells and mice that received Tcon cells or no transfer (Nil). B. Example of percentages of IFN-γ-producing CD8+ cells in response to H-2^{b} antigen determined by flow cytometry. C. Number of IFN-γ-producing splenic CD8+ cells against H-2^{b} and H-2^{k} antigens. P values were determined as described above. The experiment was repeated with similar results. D. DBA/2 mice were immunized with 10×10⁶ B6 splenocytes injected intravenously with or without 10×10⁶ D2 Treg cells. Unimmunized mice served as controls. One month later, fresh splenic T cells were tested for anti-H-2^{b} CTL activity, or alloactivated with B6 stimulator cells. The cells from the MLR cultures were re-counted and assayed for CTL activity at the indicated effector to target ratio. Values indicate the mean±SEM of 6 mice. The experiment was repeated with similar results.

FIG. 3 demonstrates that transferred anti-H-2^{b} Tregs results in the reduced cytotoxicity activity in vivo. The experimental design is similar to that described in FIG. 1. Naive DBA/2 mice were injected IV with or without 10×10⁶ D2 Tcon, Treg cells or no cells (N=8/group). On the third week, 4 mice (one half) of each group were injected with 10×10⁶ B6 splenocytes. To assess the immune response to B6 cells, one week later all mice received 10×10⁶ B6 splenocytes brightly labeled with CFSE and a similar number of dimly CFSE labeled C3H splenocytes. The animals were sacrificed 2 hours later and splenic cells examined for intensity of CFSE staining by flow cytometry. Results are expressed as the percentage of killing B6 or third party C3H CFSE stained cells in immunized animals compared with that in unimmunized animals.

FIG. 4 demonstrates that continuous antigen-stimulation results in a progressive increase in CD4+CD25+ cells and maintenance of tolerogenic effects. A. Groups of 6 mice received a single injection of 10×10⁶D2 Treg (circles), Tcon (squares) or no cells (triangles). Those with filled symbols also were also injected with 10×10⁶ H-2^{b} B6 irradiated splenocytes every two weeks. Those with empty symbols were not boosted with alloantigen. Splenic CD4+CD25+ cell numbers were determined each month by cell counts and flow cytometry in mice. Note the antigen-dependent increase in CD4+CD25+ cells in mice that received alloantigen. B. Two months after the single dose of Tcon or Tregs, some groups continued to receive specific antigen, but others were injected with third party H-2^{k} (C3H) antigen followed by another injection two weeks later. Note that the increased numbers CD4+CD25+ cells in mice given Tregs and boosted with H-2^{b} cells decreased to baseline levels when H-2^{K} cells were given instead. C. D2 mice received a single injection IV of 10×10⁶ syngeneic Tcon or Tregs, and 10×10⁶ irradiated B6 splenocytes every two weeks to provide a continuous source of antigen. One, 2, and 3 months post-injection, splenic T cells were tested for CTL activity in an allo-MLR with results in lytic units expressed as the mean±SEM. One lytic unit is the number of lymphocytes required to give 30% lysis. Six mice per group were examined at each time point. D. The tolerogenic response was antigen-dependent. Using the protocol described in the description of FIG. 4B, H-2^{k} cells were substituted for H-2^{b} cells at 2 months and the animals were tested for anti-B6 CTL activity one month later. Note the loss of CTL activity at this time that is associated with the cessation of vH-2^{b} antigen stimulation.

FIG. 5 demonstrates that CD4+CD25+ cells express increased levels of FoxP3 mRNA and protein. The experimental design is similar to that shown in FIG. 4. Groups of 4 D2 mice received a single injection of 10×10⁶ syngeneic Treg, Tcon. Another 2 mice received no T cells. All mice shown were injected with 10×10⁶ H-2^{b} B6 irradiated splenocytes every two weeks. Splenic CD4+CD25+ cell numbers of each mouse was determined at two months by cell counts and FACS staining. A. Splenic CD4+CD25+ cells were positively selected from individual mice by immunomagnetic beads, and FoxP3 mRNA was quantified by real-time PCR. The numbers shown are the mean±SEM of each group. B. A representative example of FoxP3 protein expression in these CD4+CD25+ cells was determined by staining with anti-mouse FoxP3 antibody. C. The numbers shown indicate the mean±SEM of total CD4+CD25+FoxP3+ cells of each group.

FIG. 6 demonstrates that CD4+CD25+ cells are responsible for tolerance to donor alloantigens. A. Splenic T cells, T cells depleted of CD25 prior to the culture, and CD25 depleted T cells with 10% of these CD25+ cells added back, were prepared from mice that had received a single injection of Tcon, Tregs, or no injection (No transfer) three months previously. These D2 T cells were alloactivated with B6 stimulator cells and tested for proliferative ability. Note that CD4+CD25+ cells were responsible for the suppressive effects. B. Each T cell preparation was also tested for anti-B6 CTL activity and these suppressive effects were also dependent on CD25+cells. Values shown are representative of the 6 mice in each group.

FIG. 7 demonstrates that the transferred Tregs increase recipient CD4+CD25+ cells that express CD103, CD122 and GITR. To distinguish transferred T cells from recipient T cells, anti-H-2^{d} Tregs and Tcon were prepared from cells from B6 Thy1.1 mice and 8×10⁶ cells transferred to congenic Thy 1.2 mice. Using the repeated stimulation protocol described above, the numbers of CD4+CD25+ cells and phenotype was assessed sequentially for 3 months. A. Total numbers of recipient Thy 1.2 CD4+CD25+ cells each month. B. Flow cytometry profile at 1 and 3 months of splenic cells stained with CD4, Thy1.2 and CD25. The cells shown were gated on CD4+ cells. C. Percentage of CD4 cells expressing CD25, CD122 and CD103 in the Thy 1.2 gate.

FIG. 8 demonstrates that transferred Tregs induce recipient CD4+ cells to become antigen-specific suppressor cells. A. Three months after transfer of Tcon or Tregs, splenic CD4+CD25+ and CD4+CD25− cells were obtained by cell sorting and their suppressive effects on the allogeneic response of fresh, syngeneic CD4+CD25− cells to H-2^{d}, indicated as baseline. The ratio of sorted CD4+ cells to responder CD4+CD25− cells was 1:6 to dilute out the non-specific suppressive activity of CD4+CD25+ cells (see below). The effect of neutralizing anti-IL-10 (10 μg/ml) or TGF-β (10 μg/ml) antibodies on the suppressive activity of CD4+CD25+ cells is also shown. B. Lack of suppression following stimulation by third party (H-2^{k}) cells. Results are expressed as mean cpm±SEM of triplicate wells (n=6 mice/group). C. Suppressive activity of naive CD4+CD25+ cells. CD4+CD25+ and CD25− cells from naive mice were prepared by cell sorting and assayed for their suppressive effects on the response of CD4+CD25− cells to H-₂^{b} stimulator cells.

### DETAILED DESCRIPTION OF THE INVENTION

Donor alloactivated recipient regulatory T cells (“recipient Treg cells” or “Treg cells”) are used with donor antigen to prevent rejection of transplanted tissue. As described below, recipient Tregs are prepared ex vivo by culturing recipient PBMCs with donor antigen. The recipient Tregs, with or without donor antigen, are introduced into the recipient prior to transplantation of donor tissue. Thereafter, donor antigen, alone or in combination with recipient Tregs, is administered to the recipient. This treatment prevents rejection of the transplanted tissue. It is to be understood that preventing rejection includes complete prevention as well as delayed rejection compared to transplantation without the use of donor antigen after transplantation.

Methods for making recipient Treg cells are well known in the art. (See, e.g., PCT Publication WO/01/77299 published Oct. 18, 2001, incorporated herein by reference.) Briefly, the recipient PBMCs are cultured with donor antigen in the presence of a regulatory composition. The culturing can last up to about 5-7 days after which the recipient Treg cells start to loose immunosuppressive function.

An alternate approach uses two stage culturing of the recipient PBMCs as disclosed in U.S. Patent Application 60/668,676, filed Apr. 5, 2005 incorporated herein by reference. Briefly, the methods involve: (1) removing cells from a patent and treating them for 24-48 hours with a first regulatory composition comprising TGF-β and optionally a mitogen and/or cytokine, (2) removing the first regulatory composition followed by (3) culturing the cells with a second regulatory composition comprising a cytokine. T regs produced by treatment with these two regulatory compositions produce a higher ratio of suppressor cells to helper cells as compared to treatment with TGF-β and cytokine for 5-6 days.

By “regulatory composition” herein is meant a composition that can cause the formation of regulatory T cells when cultured with recipient PBMCs and donor antigen. Generally, these compositions comprise TGFβ alone or in combination with a cytokine such as IL-2, IL-4, IL-10, IL-15 and/or TNIα. IL-2 is the preferred cytokine.

Suitable regulatory compositions may also include T cell activators such as anti-CD2, including anti-CD2 antibodies and the CD2 ligand, LFA-3, and mixtures or combinations of T cell activators such as Concanavalin A (Con A) or staphylococcus enterotoxin B (SEB). A preferred regulatory composition for antibody suppression is a mixture containing a T cell activator, IL-2 and TGF-β. In a preferred embodiment, anti-CD3 or anti-CD28 are used in combination with TGFβ and cytokine.

By “transforming growth factor-β” or “TGF-β” herein is meant any one of the family of the TGF-βs, including the three isoforms TGF-β1, TGF-β2, and TGF-β3; see Massague, J. (1980), J. Ann. Rev. Cell Biol 6:597. Lymphocytes and monocytes produce the β1 isoform of this cytokine (Kehrl, J. H. et al. (1991), Int J Cell Cloning 9: 438-450). The TFG-β can be any form of TFG-β that is active on the mammalian cells being treated. In humans, recombinant TFG-β is currently preferred. A preferred human TGF-β can be purchased from Genzyme Pharmaceuticals, Farmington, Mass. In general, the concentration of TGF-β used ranges from about 2 picograms/ml of cell suspension to about 5 nanograms, with from about 10 pg to about 4 ng being preferred, and from about 100 pg to about 2 ng being especially preferred, and 1 ng/ml being ideal.

IL-2 can be any form of IL-2 that is active on the mammalian cells being treated. In humans, recombinant IL-2 is currently preferred. Recombinant human IL-2 can be purchased from R & D Systems, Minneapolis, Minn. In general, the concentration of IL-2 used ranges from about 1 Unit/ml of cell suspension to about 100 U/ml, with from about 5 U/ml to about 25 U/ml being preferred, and with 10 U/ml being especially preferred. In a preferred embodiment, IL-2 is not used alone.

In some embodiments it is desirable to use a mitogen to activate the cells; that is, many resting phase cells do not contain large amounts of cytokine receptors. The use of a mitogen such as Concanavalin A or staphylococcus enterotoxin B (SEB) can allow the stimulation of the cells to produce cytokine receptors, which in turn makes the methods of the invention more effective. When a mitogen is used, it is generally used as is known in the art, at concentrations ranging from 1 μg/ml to about 10 μg/ml is used. In addition, it may be desirable to wash the cells with components to remove the mitogen, such as α-methyl mannoside, as is known in the art.

In a preferred embodiment, T cells are strongly stimulated with mitogens, such as anti-CD2, anti-CD3, anti-CD28 or combinations of these monoclonal antibodies especially anti-CD3 and anti-CD28. Repeated stimulation of the T cells with or without TGF-β in secondary cultures may be necessary.

A subset of CD4+ T cells that express CD25, the alpha chain of the IL-2 receptor, can induce and maintain T cell non-responsiveness to donor alloantigens and, therefore, have attractive therapeutic potential in solid organ transplantation. Peripheral CD4+ cells alloactivated with IL-2 and TGF-β ex vivo express the transcription factor FoxP3, and become potent antigen-specific suppressor cells. The transfer of TGF-β induced regulatory T cells co-incident with transplantation of a histo-incompatible heart resulted in extended allograft survival. To account for this result, non-transplanted mice were injected with a single dose of regulatory T cells and transferred donor cells every two weeks to mimic the continuous stimulation of a transplant. Increased splenic CD4+CD25+ cells were observed that were of recipient origin. These cells rendered the animals non-responsive to donor alloantigens by an antigen-specific and cytokine-dependent mechanism of action. Both the increased number of CD4+CD25+ cells and their tolerogenic effect were dependent upon continued donor antigen boosting. Thus, regulatory T cells generated ex vivo can act like a vaccine that generates host suppressor cells with the potential to protect MHC mismatched organ grafts from rejection.

As used herein, a “donor antigen” can be any antigen derived from a donor that (1) induces the formation of a recipient's regulatory T cells or (2) boosts the recipient Treg population when administered to the recipient. Examples of donor antigens include donor cells such as spleen cells, peripheral blood mononuclear cells, bone marrow cells, lymph node cells, tonsil cells and tissue extracts containing histocompatibility antigens. Other examples of donor antigens include peptides and proteins derived from the donor's major histocompatibility complex (MHC) that are produced recombinantly as well as peptides and proteins derived from related MHCs

After the MHC antigens of the donor's cells are typed, donor PBMC or histocompatible PBMC, or preferably, recombinant MHC peptides shared by the donor are cultured with recipient purified CD4+ and/or CD8+ cells in sufficient quantities to activate the recipient T cells. Activation is defined as the expression of specific surface markers or the proliferation of these cells as assessed by standard methods known to those familiar with the art. The donor cells can be used directly, or converted to antigen-presenting dendritic cells by standard methods. Ratios of donor cells to recipient cells vary between 0.01:1 (for dendritic cells) to 1:1 (for irradiated donor non-T cells). The number of Tregs transferred can range from 10⁵ to 10⁸ cells per kg. The number of donor cells used to sustain Treg activity can range from 10⁴ to 10⁷ cells per kg. Donor B cells or histocompatible B cells from a related donor can be greatly expanded by EBV transformation and used as the source of donor antigen.

As used herein, “donor tissue” is any tissue that can be transplanted from one individual to another, preferably within the same species. Donor tissue includes kidney, heart, lung, liver, intestine, pancreas and pancreatic islet cells. The preferred recipient is human.

### EXAMPLES

Tregs induced ex vivo can substantially delay rejection of heart allografts in non-lymphopenic mice using allogeneic spleen cell immunization. The transfer of TGF-β induced Tregs have antigen-specific tolerogenic effects in these mice. These cells induced recipient CD4+ cells to become CD4+CD25+ cells that are responsible for the T cell non-responsiveness. In order to sustain these CD4+CD25+ cells and their tolerogenic effects, continuous boosting of allogeneic donor cells was required.

Materials and Methods

Animals

Male C57BL/6 (B6, H-2^{b}), DBA/2 (D2, H-2^{d}), and C3H (H-2^{k}) mice were purchased from the Jackson Laboratory (Bar Harbor, Me.). Animals eight to ten weeks of age were used as graft donors, recipients, and controls. All mice were housed in conventional facilities at University of Southern California using animal care protocols approved by the IACUC of University of Southern California.

Antibodies and Reagents

The following Abs were obtained from eBioscience (San Diego, Calif.): Anti-CD3-PE (145-2011), anti-CD4-FITC (RM4-5), anti-CD4-PE (GK1.5), anti-CD8-PE (53-6.7), anti-CD25-PE (PC61), anti-CTLA-4-PE (UC10-4B9), anti-CD122-PE (51-14), anti-CD103-FITC (2E7), anti-IFN-γ (XMG1.2), anti-FoxP3 (FJK-16S), anti-Thy1.1-PE (A20) and anti-Thy1.2-FITC (104). The anti-H-2^{d}-FITC (SF1-1.1) and anti-H-2^{b} (AF6-88.5) came from BD Pharmingen (San Diego, Calif.). Isotype controls Abs were also obtained from eBioscience and BD Pharmingen. Anti-GITR-biotin (BAF524), anti-IL-10 (mAb417), anti-TGF-β (mAb240) and matched isotype control abs were obtained from R&D Systems (Minneapolis, Minn.).

Cell Preparation and Adoptive Transfer

T cells were prepared from D2 spleen cells by collecting nylon wool column non-adherent cells (Zheng, S. G., et al., J Immunol 172:1531 (2004)). The T enriched cells (1.5×10⁶ per ml) were stimulated with similar numbers of irradiated (2000 rad) B6 nylon adherent, non-T cells for 5-6 days in 24 well plates (2 ml/well) (Becton Dickinson Labware, Franklin Lakes, N.J.) in AIM V (InVitrogen, Carlsbad, Calif.) serum-free medium with additives (Zheng, S. G., et al., J Immunol 172:1531 (2004)). Some wells contained TGF-⊕1 (2 ng/ml) and rhuIL-2 (15 to 20 units/ml) (R&D Systems) or IL-2 only. Groups of 6 D2 mice were injected intravenously 1 day before and 5 days after receiving B6 heart allograft with ten million viable alloactivated T cells primed with IL-2 and TGF-β (Treg) and others with IL-2 only (Tcon), or with Treg depleted of CD25+ cells with immunomagnetic beads (Miltenyi). These preparations contained approximately 10% residual B6 stimulator cells.

Heterotopic Heart Transplantation

Abdominal vascularized heterotopic heart transplants were performed essentially as previously described (Cramer, D. V., et al. In Handbook of Animal Models in Transplantation Research, 1st edn., p. 149-160. CRC Press, Boca Raton, La. (1993)). Rejection was defined as complete cessation of a palpable cardiac contraction and confirmed by visualization after laparotomy. Recipients with grafts surviving >100 days were considered as permanent and were sacrificed for in vitro experiments.

Assays of T Cell Function

The proliferative activity of T cells to alloantigens was measured using a standard one way mixed lymphocyte culture with 2×10⁵ T cells and an equal number of irradiated allogeneic non-T cells in a 96 well flat bottomed plate using RPMI 1640 culture medium and 10% fetal calf serum with additives as described previously (Zheng, S. G., et al., J Immunol 172:1531 (2004)). Proliferation was measured after 4-5 days as uptake of ³H-thymidine in triplicate cultures. In order to analyze the IFN-γ-producing cells, intracellular cytokine staining was performed as described previously (Zheng, S. G., et al., J Immunol 172:5213 (2004)). In cultures used to assess the suppressive activity of CD4+CD25+ cells, the ratio of primed cells to CD4+CD25− responder cells was 1:6. T cell cytotoxic activity was assessed using various ratios of effector cells to target cells (Chromium-labeled Con A blasts) in a standard 4 hour assay as described previously. Values indicate the mean±SEM of triplicate cultures and in some experiments expressed as the lytic units per 10⁶ cells (Yamagiwa, S., et al., J Immunol 166:7282 (2001)). Lytic units were based on the number of effector cells required to kill 30% of the target cells.

FoxP3 Expression by Real-Time RT-PCR

Total RNA was prepared with TRIzol LS reagent (Invitrogen). First strand cDNA was synthesized using Omniscript TR kit (Qiagen, Valencia, Calif.) with random hexamer primers (Invitrogen). Real-time PCR was performed with a LightCycler (Roche, Mannheim, Germany), and message levels were quantified using the LightCycler Fast Start DNA Master SYBR Green I Kit (Roche), according to the manufacturer's instructions. Amplification was conducted for 45 cycles. The recovered PCR product and amplicon were checked by agarose gel electrophoresis for a single band of the expected size. The samples were run in triplicate and the relative expression of FoxP3 was determined by normalizing expression of each target to hypoxanthine guanine phosphoribosyl transferase (HPRT). Primer sequences were as follows: HPRT 5′-TGA AGA GCT ACT GTA ATG ATC AGT CAA C-3′ (SEQ ID NO:1) and 5′-AGC AAG CTT GCA ACC TTA ACC A-3′ (SEQ ID NO:2); FoxP3 primers: 5′-CCC AGG AAA GAC AGC AAC CTT-3′ (SEQ ID NO:3) and 5′-TTC TCA CAA CCA GGC CAC TTG-3′ (SEQ ID NO:4) (Hori, S., Nomura, T., and Sakaguchi, S. Science 299:1057 (2003)).

In Vivo Cytototoxic T Cell Activity

Groups of 8 DBA/2 mice were injected intravenously with 10⁷ Treg or T con cells generated ex vivo as described above. Another group was not injected. Three weeks later, four mice from each group were injected with 10⁷ C57BL/6 splenocytes (immunized) or served as controls. In vivo cytotoxic T cell activity was assessed at week four using an assay modified from that described by Suvas and co-workers (Suvas, S., et al., J Exp Med 198:889 (2003)). Splenic target cells from C57BL/6 or C3H mice were labeled with high (2.5 mM) or low (0.25 mM) concentrations of CFSE. Equal numbers (10⁷) of donor-specific and third party target cells were mixed together and adoptively transferred intravenously into control and immunized DBA/2 mice. Splenocytes were collected at 1, 2 or 4 h after adoptive transfer from recipient mice, erythyrocytes were lysed, and cell suspensions were analyzed by flow cytometry. Each population could be distinguished by their respective fluorescence intensity. Assuming that the number of C57BL/6 target cells that migrated to the spleen in unimmunized mice is equivalent to the number of splenic C57BL/6 target cells injected in immunized mice, the percentage of killing of target cells in the immunized animals was determined as: % Killing=[(Percentage of CFSE⁺ subset in the control mice−percentage of CFSE⁺ in the immunized mice)÷Percentage CFSE⁺ in the control mice]×100.

Statistical Analysis

Analysis for statistically significant differences between groups of mice was performed by t test and Wilcoxon test survival curves with the log rank test using GraphPad PRISM software (GraphPad, San Diego, Calif.).

Results

Treatment with Regulatory T Cells Generated Ex Vivo Markedly Prolongs the Survival of Heart Allografts

Since we have shown that TGF-β induces both CD4+ and CD8+ cells to become suppressor cells (Yamagiwa, S., et al., J Immunol 166:7282 (2001); Gray, J. D., et al., J Exp Med 180:1937 (1994), and others have described CD8+ regulatory cells that express FoxP3 with functional properties similar to CD4+CD25+ regulatory T cells (Xystrakis, E., et al., Blood 104:3294 (2004)), we generated Tregs from unseparated T cells. Our objective was to learn whether a combination of CD4+ and CD8+ Tregs induced ex vivo with TGF-β used as sole therapy could prolong survival of totally MHC mismatched heart allografts. After culture of DBA/2 (H-2^{d}) T cells with irradiated C57BL/6 (H-2^{b}) spleen cells for 5 to 6 days with IL-2 and TGF-β, we recovered approximately the starting number of T cells in cultures with TGF-β, and 50% of T cells in cultures without TGF-β. In cultures with IL-2 and TGF-β, 60±4.1% of CD4+ cells expressed CD25 and 55±4.8% of CD8+ cells expressed this marker. These cells are called Treg. In cultures without TGF-β these values were 45±3.4% and 49±4.1% respectively. These cells are called Tcon. Of the 10 million cells injected into recipient mice, Treg preparations contained 3.4±0.3×10⁶ CD4+CD25+ cells and 2.1±0.2×10⁶ CD8+CD25+ cells. Tcon preparations contained 2.1±0.2×10⁶ CD4+CD25+ cells and 1.6±0.15×10⁶ CD8+CD25+ cells, respectively.

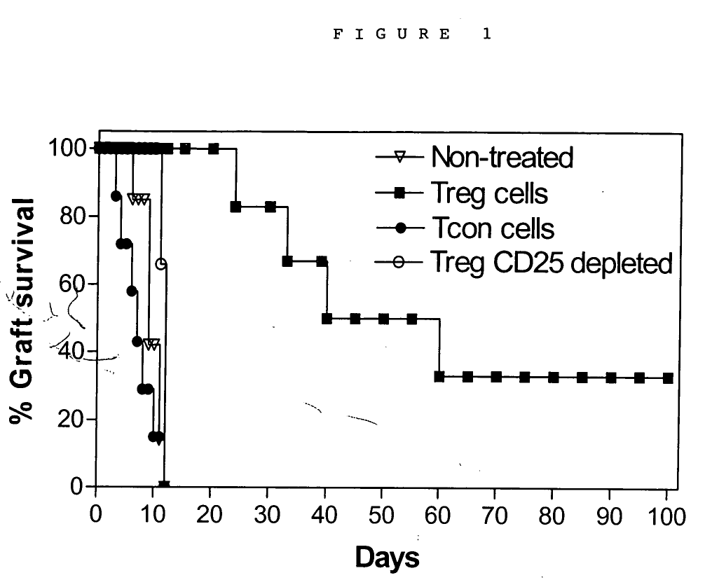

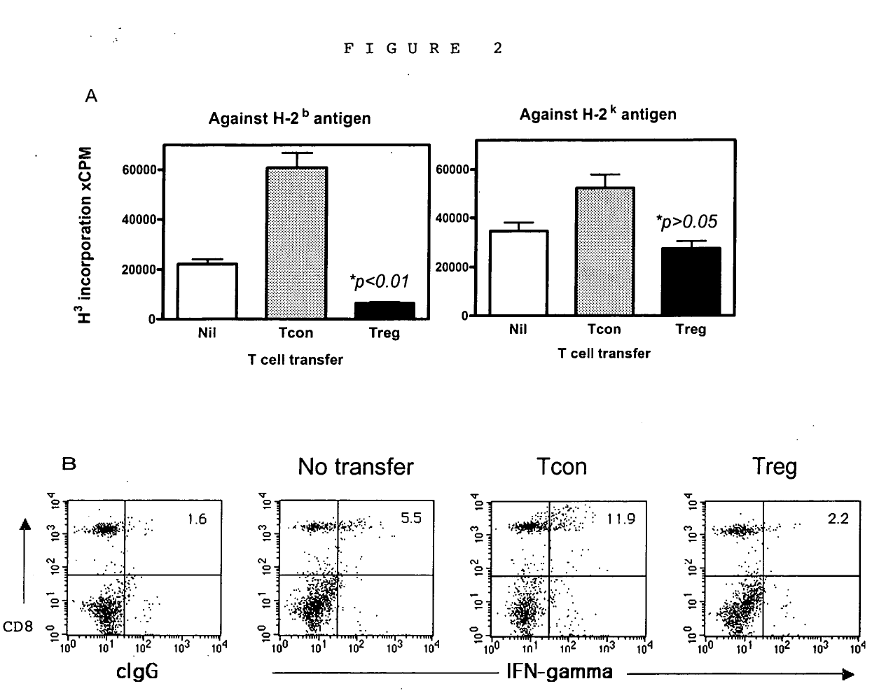

All hearts from B6 mice that were transplanted into D/2 recipients were rejected within 11 days of transplantation. Transfer of 10 million Treg at days −1 and +5 resulted in extended survival of B6 heterotopic heart transplants up to 100 days, at which point the experiment was terminated. By contrast, rejection was accelerated in D2 mice that received similar numbers of Tcon (FIG. 1). The extended survival was dependent on CD25+ cells, since depletion of this subset completely abolished all suppressive effects.

The Transfer of Treg Cells Results in the Antigen-Specific Tolerance in the Recipients

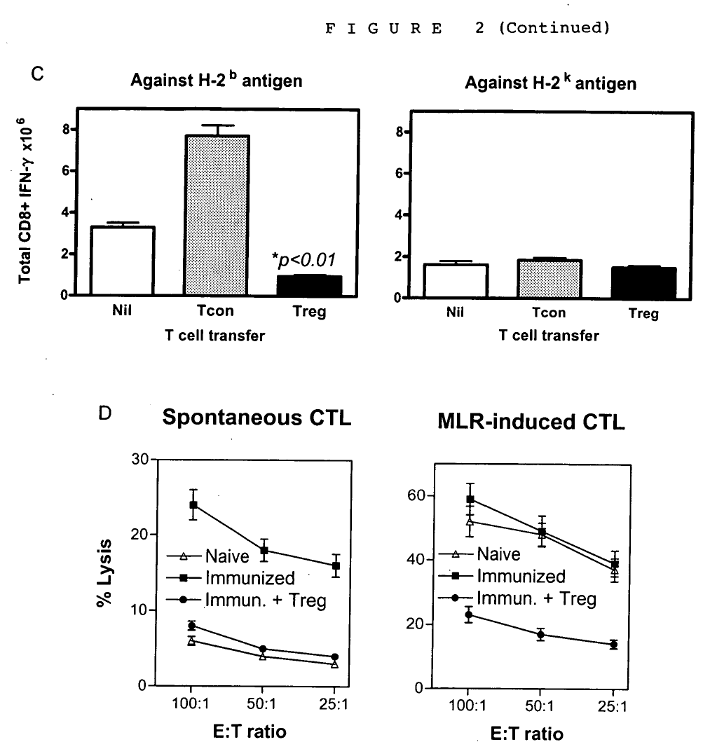

We next developed a model designed to investigate the mechanism of action of the long term suppressive effects. D2 mice were given a single injection of 10⁷ Treg or Tcon cells. One month later they were tested for T cell responsiveness to donor alloantigen. FIG. 2 shows that animals injected with Tcon proliferated vigorously to H-2^{b} antigen. By contrast, animals injected with Treg cells were non-responsive. They were unable to proliferate when challenged with alloantigen (FIG. 2A). CD8+ cells were unable to produce IFN-γ (FIGS. 2B and 2C), and were unable to kill H-₂^{b} target cells even after further stimulation in vitro (FIG. 2D). This T cell non-responsiveness was antigen-specific. D2 T cells proliferated strongly in response to third party C3H H-2^{k} stimulator cells (FIG. 2A).

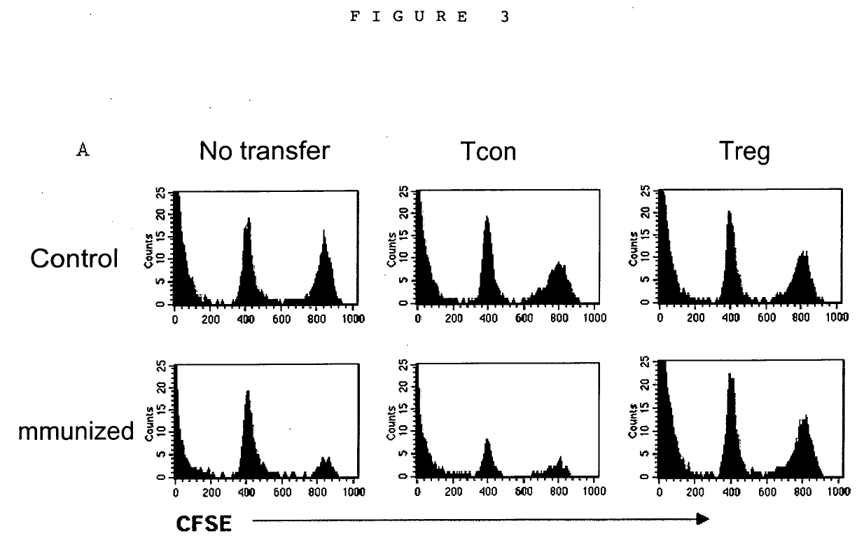

In addition to documenting T cell non-responsiveness in vitro effects, we observed similar effects in vivo. Following transfer of Treg cells previously primed with H-2^{b} alloantigen, and then boosted with donor cells, mice were injected with CFSE-labeled donor and third party target cells and examined for the presence of these cells in the spleen. Pilot studies revealed that following immunization, there was a marked reduction of donor, but not third party target cells within 2 hours of injection (FIG. 3A). However, in mice that had received Treg, similar numbers of CFSE-labeled donor target cells were observed in control and immunized mice. By contrast, in mice that had received Tcon, numbers of both donor and third party targets were markedly reduced. The reduction of third party target cells probably reflects the non-specific CTL activity associated with the vigorous CTL response to donor alloantigen. Table I indicates that the effects we observed were very similar in the 4 mice of each group. Since the in vivo CTL assay does not require the in vitro expansion, this approach is considered to be direct evidence of Treg function in vivo (Suvas, S., et al., J Exp Med 198:889 (2003)).

TABLE I

| | |
|---|---|
| Mice (n = 4/group) | |
| Immunized vs | Target Cells |

| Naïve mice | H-2^{b} (CFSE bright) | H-2^{k} (CFSE dim) |
|---|---|---|
| No transfer | 74.4 ± 4.3%** | 0.8 ± 0.02% |
| Tcon | 75.9 ± 4.1%** | 61.2 ± 6.6%* |
| Treg | 0.2 ± 0.006% | 0.5 ± 0.01% |

The values shown indicate the killing of CFSE-labeled H-2^{b} or H-2^{k} target cells in the spleens of immunized mice determined by a formula indicated in materials and methods.
The p values indicate significant differences between mice injected with Treg or T control, or immunized mice that did not receive cell transfer.
*indicates p < 0.01),
**indicates p values < 0.001)

T Cell Non-Responsiveness Depends upon CD4+CD25+ Cells that Require Continuous Specific Antigen Stimulation

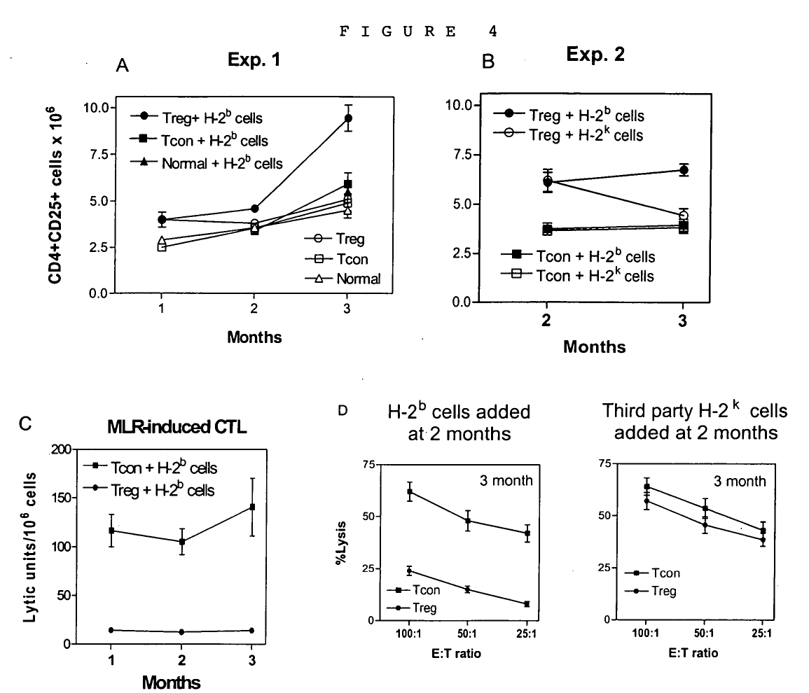

The next series of experiments confirmed the requirement of CD4+CD25+ cells for the suppressive effects and revealed that continuous stimulation of specific antigen was needed to sustain T cell non-responsiveness. Groups of mice received a single injection of Treg or Tcon, or no cells. Some mice received booster injections of donor alloantigen every two weeks and others not injected served as controls. In animals that had received the booster injections, we observed a progressive increase in the splenic CD4+CD25+ cells during the next three months in animals that had received Tregs, but not in those that had received Tcon cells (FIG. 4A). Since these mice were not lymphopenic, the increase could not be attributed to the homeostatic expansion of CD4+CD25+ cells described by others (Annacker, O., et al., J Immunol 166:3008 (2001)). This expansion was dependent upon continuous boosting with donor alloantigen. If at two months the mice received splenic cells from H-2^{k} C3H mice instead of H-2^{b} B6 cells, the numbers of CD4+CD25+ cells decreased to baseline values within one month (FIG. 4B). Splenic CD8+CD25+ cells probably did not play a significant role since they comprised <1% of CD8+ cells in mice that had received Treg.

Continuous stimulation with specific antigen was required for Treg to sustain blockade of CTL activity. FIG. 4C shows that the animals that had received Tregs and 3 to 5 subsequent booster immunizations of donor alloantigen for 2 to 3 months were unable to develop anti-H-2^{b} CTL activity. However, if injections of third party H-2^{k} cells instead of donor cells were given, the mice demonstrated strong anti-H-2^{b} CTL activity within one month (FIG. 4D).

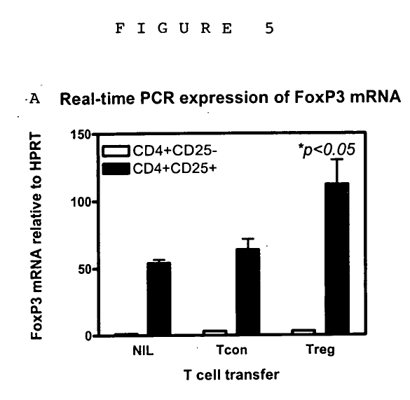

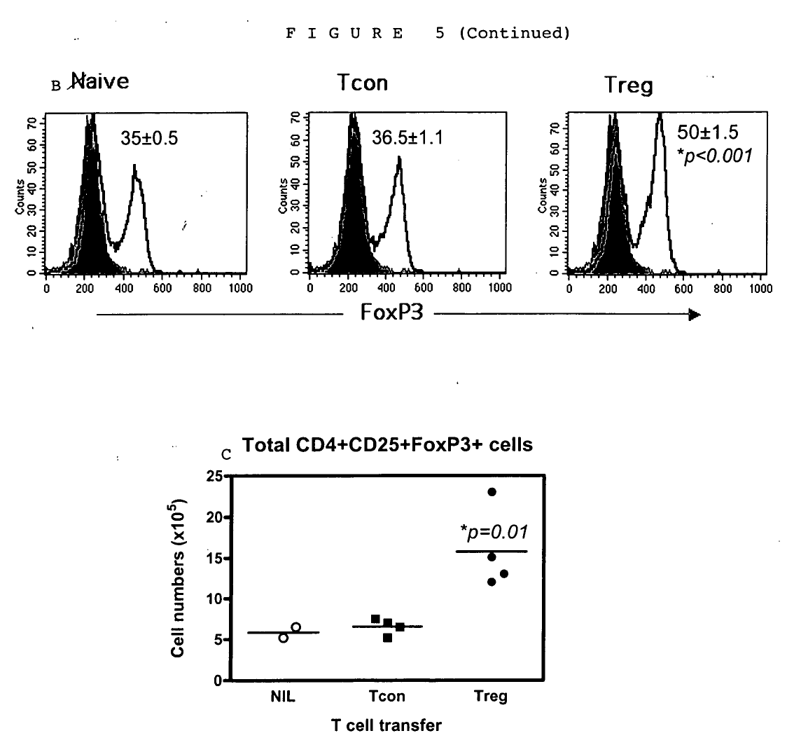

We next obtained evidence that the increased numbers of CD4+CD25+ cells in mice given Treg followed by booster immunizations of donor alloantigen expressed FoxP3 and were required for T cell non-responsiveness. Mice that received Treg, Tcon or no cells followed by booster immunizations every two weeks were sacrificed at 2 months. Although the total numbers of splenic CD4+ cells were similar in each of the groups, the CD4+CD25+ subset was significantly increased in mice that had received Treg (Table II). Examination of CD4+CD25+ and CD4+CD25− cells revealed that the CD25+ subset expressed significantly higher levels of FoxP3mRNA by real time PCR (FIG. 5B). Moreover, the number of CD4+CD25+ FoxP3+ cells quantified by flow cytometry was significantly increased in mice that had received Treg compared to those that received Tcon (FIGS. 5C and 5D).

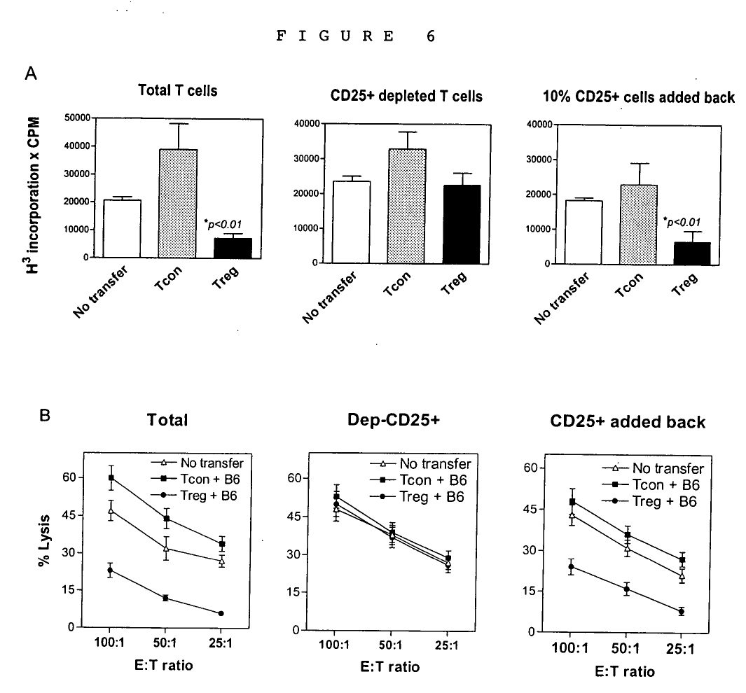

CD4+CD25+ cells were probably responsible for antigen-specific non-responsiveness to B6 alloantigens. As shown in FIG. 6A, depletion of CD25+ cells abolished the tolerogenic effect and adding back this subset restored the suppression. As with CTL activity, depletion of this CD25+ cells increased allo-CTL activity to levels similar to animals that had received Tcon. Again, adding back CD25+ Tregs in a 1:10 ratio restored suppressive activity (FIG. 6B). Since CD8+CD25+ cells comprised only 1% of total CD25+ cells, this suppressive effect was presumably to CD4+CD25+ cells. These experiments, however, do not exclude an effect of CD8+ suppressor cells.

TABLE II
CD4+CD25+ cells at two months following cell transfer

| | Spleen cells × 10⁶ | % CD4/spleen | % CD25/CD4+ | Total numbers of |
|---|---|---|---|---|
| T cell transfer | (Mean ± SEM) | (Mean ± SEM) | (Mean ± SEM) | CD4+CD25+ × 10⁶ |
| Nil (n = 2) | 89.5 ± 3.5 | 25 ± 2.0 | 7.5 ± 0.5 | 1.8 ± 0.2 |
| Tcon cells (n = 4) | 94 ± 3.2 | 28.5 ± 2.3 | 7.4 ± 0.7 | 1.9 ± 0.2 |
| Treg cells (n = 4) | 89.8 ± 3.1 | 27.7 ± 1.9 | 12.5 ± 1.1 | 3.3 ± 0.5 |
| | | | | (*p < 0.01) |

Donor Regulatory T Cells Educate Recipient T Cells to Become Tolerogenic CD4+CD25+ Cells In Vivo

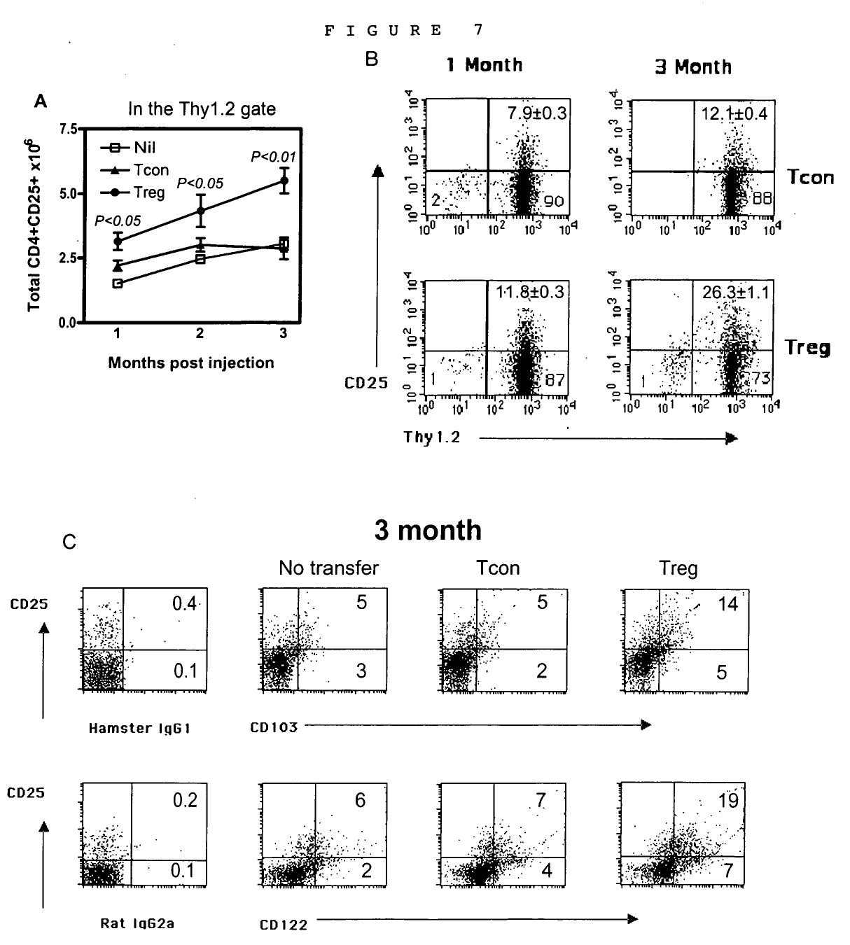

To learn whether the increased CD4+CD25+ suppressor cells were the progeny of donor Tregs or derived from the recipient, the experiment was repeated using the protocol described above with Thy1.1 B6 mice serving as the source of the Tregs and congenic Thy1.2 mice as recipients. Here we again noted a progressive increase in CD4+CD25+ cells in B6 mice during the three months following a single injection of 8 million anti-H-2^{d} Treg and almost all cells were recipient Thy 1.2 origin (FIG. 7A). At one month only 1% of splenic T cells were stained by anti-Thy1.1 (results not shown). Thy 1.2 negative T cells were <2% and these cells did not express CD25 (FIG. 7B). In comparison with Tcon, Tregs were enriched in cells expressing CD25, CD122 (IL-2R chain), CD103 (alpha E integrin) and GITR (FIG. 7C and 7D.), and most of the CD122 and CD103 cells also expressed CD25 (FIG. 7C). See also Table III. Others have shown that TGF-β up-regulates CD103 expression (Cerwenka, A., et al., J Immunol 153:4367 (1994)).

TABLE III
Phenotypic characterization of CD4+ cells at three months following T cell transfer

| T cell transfer | | | | |
|---|---|---|---|---|
| (n = 3) | % CD25/CD4 | % CD122/CD4 | % CD103/CD4 | % GITR/CD4 |
| Nil | 12.3 ± 0.9 | 8 ± 0.5 | 7.9 ± 0.9 | 12.5 ± 0.8 |
| Tcon cells | 12 ± 1.2 | 10.2 ± 0.4 | 7.2 ± 0.5 | 15.6 ± 1.2 |
| Treg cells | 25.7 ± 1.0 (***) | 25.3 ± 0.6 (***) | 19.6 ± 1.3 (**) | 29.5 ± 5.3 (*) |

(*) indicates p < 0.05;
(**) p < 0.01;
(***) p < 0.001
Mean ± SEM % CD4+ cells expressing CD25, CD122, CD103 and GITR in the Thy 1.2 gate (6 mice per group).
P values indicate significant differences between Treg and Nil or Tcon.

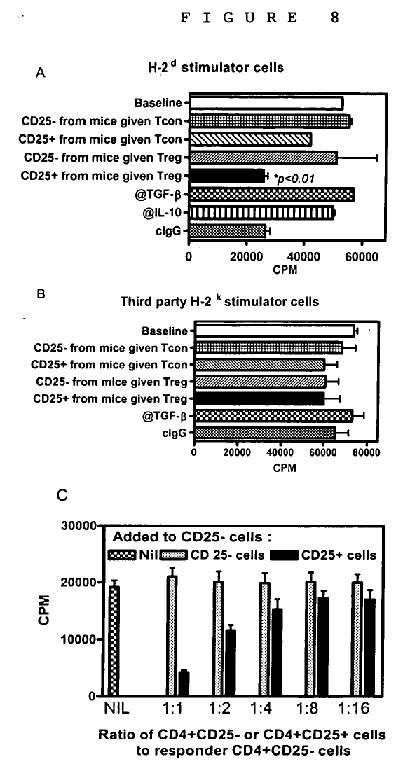

The functional properties of the educated mouse splenic CD4+CD25+ cells were similar to educated human peripheral blood CD4+ cells reported previously (Zheng, S. G., et al., J Immunol 172:5213 (2004)). While natural CD4+CD25+ cells and human natural-like CD4+CD25+ cells induced with TGF-β have cytokine-independent suppressive activity (Sakaguchi, S., et al., Immunol Rev 182:18 (2001); Piccirillo, C. A. and Shevach, E. M., Semin Immunol 16:81 (2004); Yamagiwa, S., et al., J Immunol 166:7282 (2001)), the suppressive activity of our educated CD4+CD25+ cells was abolished by either anti-TGF-β or anti-IL-10 (FIG. 8A). Of great interest, the anti-H-2^{d} suppressive activity of CD4+CD25+ cells from mice that had received Tregs was significantly greater than CD4+CD25+ cells from mice that had received Tcon. This effect was antigen-specific since anti-H-2^{d} CD4+CD25+ cells had minimal suppressive activity against H-2^{k} stimulator cells (FIG. 8B). These experiments were performed with a single source of CD25− responder cells and a ratio of CD4regs to CD4 responders of 1:6. At this ratio, the antigen non-specific suppressive activity of endogenous CD4+CD25+ regulatory cells on the response of CD4+ cells to allogeneic stimulator cells is diluted out (FIG. 8C).

Discussion

In this study we observed that Tregs induced with IL-2 and TGF-β ex vivo can prolong the survival of heart allografts in completely MHC-mismatched mice without any additional immunosuppression. We also administered repeat allogeneic cell infusions in non-transplanted mice to investigate the mechanism of action. We observed that a single injection of T cells primed with allogeneic cells and TGF-β (Treg) followed by continuous boosts of alloantigen could induce long term antigen-specific non-responsiveness in the recipients. This tolerogenic effect appeared to be secondary to the ability of the transferred Tregs to educate donor CD4+ cells to become CD25+ cells.

It is recognized that TGF-β can induce both CD4+ and CD8+ cells to become suppressor cells. In 1994 we reported that human CD8+ cells activated with IL-2 and TGF-β became cytokine-dependent suppressors of T cell-dependent antibody production (Gray, J. D., et al., J Exp Med 180:1937 (1994)). We subsequently observed that TGF-β induced naive CD4+ cells to become CD4+CD25+ cells with a phenotype and suppressive activities indistinguishable from the natural CD4+CD25+ cells described by others (Sakaguchi, S., et al., Immunol Rev 182:18 (2001); Piccirillo, C. A. and Shevach, E. M., Semin Immunol 16:81 (2004)). These cells had a contact-dependent, cytokine-independent mechanism of action and were potent inhibitors of CD8+ T cell activation (Yamagiwa, S., et al., J Immunol 166:7282 (2001); Piccirillo, C. A. and Shevach, E. M., J Immunol 167:1137 (2001)). We observed that TGF-β did not expand endogenous CD4+CD25+ cells, but induced CD4+CD25− cells to develop this function (Zheng, S. G., et al., J Immunol 172:5213 (2004)). Subsequently, Chen and co-workers reported that TGF-β induced mouse CD4+CD25− cells to become CD25+ suppressor cells that express FoxP3 (Chen, W., et al., J Exp Med 198:1875 (2003)), and our lab and others have confirmed this finding (Zheng, S. G., et al., J Immunol 172:5213 (2004); Fu, S., et al. Am J Transplant 4:1614 (2004); Schramm, C., et al., Int Immunol 16:1241 (2004); Park, H. B., et al., Int Immunol 16:1203 (2004); Fantini, M. C., et al., J Immunol 172:5149 (2004)). Prior work by Blazar and co-workers found that CD4+CD25− cells tolerized with IL-2 and TGF-β could increase survival in a model of alloantigen-induced graft versus host disease (Chen, Z. M., et al., Blood 101:5076 (2003)).

Since we had been able to induce CD8+ cells to become suppressor cells with TGF-β, and others had shown that CD8+ regulatory cells could suppress the rejection of heart allografts (Liu, J., et al., Transpl Immunol 13:239 (2004)), we utilized total T cell preparations containing both CD4+ and CD8+ cells in our initial study. In preliminary work using the mouse graft-versus-host disease model that we had used previously (Zheng, S. G., et al., J Immunol 172:1531 (2004), we found that the combination of CD4+ and CD8+ Tregs have more potent therapeutic effects than purified CD4+ Tregs (unpublished observations). In the present experiments, we observed only small numbers of CD8+CD25+ cells in the recipients, and less than 1% Thy1.1 congenic T cells remained in the recipients one month after transfer. Nonetheless, we cannot exclude the possibility that CD8+ Tregs induced with TGF-β contributed to the observed therapeutic effects.

In this study the number of CD4+CD25+ cells of recipient origin progressively increased in response to bi-weekly booster immunizations with allogeneic cells. Although CD25 is a marker of activated T cells, it is unlikely that the cells we observed are allogeneic effector cells. These cells were non-responsive to donor alloantigen. They blocked the ability of recipient T cells to proliferate and produce cytokines in response to donor alloantigens and they prevented CD8+ cells from developing CTL activity. Furthermore, the persistence of donor target cells in the in vivo CTL assay provides additional evidence of Treg activity in vivo as stated above. Finally, the evidence that these CD4+CD25+ cells express both FoxP3 mRNA and protein strongly suggests that the booster immunizations were expanding CD4+CD25+ regulatory cells in vivo.

Our results are consistent with other reports that CD4+CD25+ regulatory cells have a protective effect in transplant rejection. Van Maurik and colleagues have documented that CD4+CD25+ can markedly prolong survival of cardiac allografts, although these cells were induced by indirect methods (van Maurik, A., et al., J Immunol 169:5401 (2002)). Benghiat and co-workers have recently reported that natural CD25+ Treg cells control Th1- and Th2-type allo-helper T-cell responses (Benghiat, F. S., et al., Trnasplantation 79:648 (2005)). Both of these groups have reported that the protective CD4+CD25+ cells require continuous antigen stimulation (Cobbold, S. P., et al., Transpl Int 16:66 (2003); Thorstenson, K. M. and Khoruts, A., J Immunol 167:188 (2001)). Schenk and co-workers have reported that depletion of CD4+CD25+ cells markedly accelerates acute rejection of heart allografts rejection (Schenk, S., et al., J Immunol 174:3741 (2005)). Because epitope spreading in alloreactive cells may contribute to chronic rejection (Ciubotariu, R., et al., J Clin Invest 101:398 (1998)), Salama and co-workers have suggested that CD4+CD25+ cells may limit this effect and thus have a protective role (Salama, A. D., et al., J Am Soc Nephrol 14:1643 (2003)).

While some research has shown that polyclonal CD4+CD25+ cells can educate other CD4+ cells to become suppressor cells in vitro (Jonuleit, H., et al., J Exp Med 196:255 (2002); Dieckmann, D., et al., J Exp Med 196:247-53 (2002)), others have used indirect methods to achieve infectious tolerance in vivo (Qin, S., et al., Science 259:974 (1993)). This is the first demonstration that Tregs induced ex-vivo can educate recipient CD4+ cells to become CD25+ cells that have similar suppressive activity. Thus, this TGF-β induced regulatory T cells act more like a vaccine than a conventional adoptive therapy. They appear to prevent organ graft rejection by eliciting an active, protective immune response in the recipient.

Examination of the functional properties of CD4+CD25+ cells harvested from the tolerized recipients revealed that their mechanism of action could be blocked by either anti-TGF-β or anti-IL-10. This result is consistent to a study of human CD4+CD25+ regulatory cells induced with TGF-P. We reported that anti-TGF-β was unable to block the suppressive effects of naive CD4+ cells induced ex-vivo to become alloantigen-specific suppressor cells. Nonetheless, these cells produced both TGF-β and IL-10 following restimulation, and both of these cytokines were necessary for these CD4+CD25+ Tregs to induce other CD4+CD25− to become suppressor cells. Moreover, the suppressive effects of the secondary CD4+CD25+ Tregs were blocked by either anti-TGF-β or anti-IL-10. Thus, the transfer of CD4+CD25+ Tregs with cytokine-independent suppressive effects in vitro may result in cytokine-dependent suppressive effects in vivo. In experimental models of immune-mediated disease in mice, the role of TGF-β and IL-10 in supporting the suppressive effects of CD4+CD25+ cells has been established (Coombes, J. L., et al., Immunol Rev 204:184 (2005); Peng, Y., et al., Proc Natl Acad Sci USA 101:4572 (2004)).

In this study we observed long term survival in some, but not all, of the allogeneic heterotopic heart transplants. Although we observed prolonged graft survival, only two of these six grafts survived to 100 days, making it highly unlikely that in vivo tolerance was achieved. By contrast, animals that received booster immunizations of donor alloantigen could not mount a response to donor cells. The results of these experiments infer that in the attempt to establish a tolerant state, there is a probable requirement for persistent antigenic stimulation to sustain the activity of the Tregs which may not sufficiently exist after heart transplantation alone with a presumed paucity of donor antigens being shed. Histology of the hearts from animals sacrificed 100 days post-transplantation did not show classic pathologic evidence of acute or chronic rejection. However, despite intact function, the myocardium did have a moderate monocytic infiltrate. Unfortunately, we did not save frozen tissue at the time of the original experiments, so that we cannot evaluate if there was FoxP3 mRNA displayed by these mononuclear cells, and therefore cannot exclude the possibility that this mononuclear infiltrate may be an atypical manifestation of chronic rejection. Further experiments in which animals receive additional injections of relevant donor MHC alloantigens may improve the graft survival results observed, as well as decrease the observed mononuclear infiltrates. The use of TGF-β treated T cells generated ex-vivo to alter the recipient's immune system to develop a dominant regulatory response rather than an alloreactive one offers a novel therapeutic strategy for clinical organ transplantation.

What is claimed is:

1. A method for preventing rejection of donor tissue in a recipient comprising:
(a) producing, ex vivo, a population of donor alloactivated regulatory recipient T cells (Tregs);
(b) introducing a first dose of said recipient Tregs into said recipient;
(c) transplanting donor tissue into said recipient; and
(d) introducing donor antigen into said recipient after said transplantation.

2. The method according to claim 1 wherein said first dose of recipient Tregs is introduced at least one day before transplantation.

3. The method according to claim 1 wherein said donor antigen is introduced to said recipient on a periodic basis.

4. The method according to claim 1 wherein said donor antigen is introduced on a periodic basis.

5. The method according to claim 1 wherein said donor antigen is a histocompatability antigen.

6. The method according to claim 1 wherein a second dose of recipient Tregs is introduced after transplantation.

7. The method according to claim 1 wherein a second dose of recipient Tregs is introduced from one to five days after transplantation.

8. The method according to claim 1 wherein the producing of said Tregs comprises: isolating recipient peripheral blood mononuclear cells (PBMC); and contacting ex vivo said recipient PBMCs with donor antigen and a regulatory composition comprising TGFβ to form said recipient Tregs.

9. The method according to claim 8 wherein said regulatory composition further comprises IL-2, IL-4, IL-10 and/or IL-15.

10. The method according to claim 8 wherein donor cells are used as said donor antigen.

11. The method according to claim 10 wherein said donor cells are not T cells.

12. The method according to claim 8 wherein donor PBMCs are used as said donor antigen.

13. The method according to claim 10 wherein said donor cells comprise spleen cells or irradiated PBMCs.

14. The method according to claim 1 wherein said donor tissue is selected from the group consisting of heart, lung, liver, kidney, intestine pancreas and pancreatic islet cells.

15. The method according to claim 14 wherein said donor tissue is a heart.

16. The method of claim 1 where said introducing of step (d) further comprises introducing a second dose of said recipient Tregs into said recipient.

17. A method for preventing rejection of a heart transplant comprising:
(a) producing a population of donor alloactivated recipient regulatory T cells (Tregs);
(b) introducing said recipient Tregs into said recipient;
(c) transplanting a heart from a said donor into said recipient; and
(d) introducing donor antigen into said recipient after said transplantation.

18. The method according to claim 17 wherein said producing comprises: isolating recipient peripheral blood mononuclear cells (PBMC); and contacting ex vivo said recipient PBMCs with donor antigen and a regulatory composition comprising TGFβ to form said recipient Tregs.

19. The method according to claim 16 wherein said regulatory composition further comprises IL-2.

20. The method of claim 1 or 17 wherein said recipient is a human.
